(19) United States
(12) Patent Application Publication
(10) Pub. No.: US 2013/0261412 A1
(43) Pub. Date: Oct. 3, 2013

(54) SYSTEM AND METHOD FOR MANAGING FLUID LEVELS IN PATIENTS BASED UPON CREATININE CLEARANCE

(71) Applicant: Robert Lawrence Reed, II, Indianapolis, IN (US)

(72) Inventor: Robert Lawrence Reed, II, Indianapolis, IN (US)

(21) Appl. No.: 13/852,382

(22) Filed: Mar. 28, 2013

Related U.S. Application Data
(60) Provisional application No. 61/616,527, filed on Mar. 28, 2012.

(51) Int. Cl. A61B 5/20

(52) U.S. Cl. CPC A61B 5/20
USPC 600/309

(57) ABSTRACT

A creatinine clearance monitoring and fluid level management system for use in the treatment of patients. The system establishes and adjusts the dosing of intravenous fluids based upon periodic creatinine clearance calculations based upon a system specified frequency. Warning or alert messages or signals are produced if creatinine clearance levels indicate the need for the administration of additional fluids based upon a creatinine clearance result below an established normal threshold. Furthermore, the system generates a warning to trigger a more serious intervention in the event a patient's creatinine clearance rate falls below a lower established threshold or the system determines that an inordinate amount of fluids have been administered without the anticipated response. The creatinine clearance monitoring and fluid level management system is particularly useful for patients in a hospital or in-patient environment, and particularly those post-operative patients or those in intensive care.

### CROSS REFERENCE TO RELATED APPLICATIONS

The present application claims the benefit of U.S. Provisional Patent Application Ser. No. 61/616,527 filed Mar. 28, 2012 which is hereby incorporated by reference in its entirety to the extent not inconsistent.

### FIELD OF THE INVENTION

The invention relates to the maintenance of fluid levels in patients, and in particular, to a system that aids in the administration of fluids to patient and monitors for an indication that immediate intervention is necessary through the use of computerized fluid calculations that are made with the use of creatinine clearance rates.

### BACKGROUND

Maintaining a proper fluid level is important for many patients, particularly post-op and critical care patients, in order to prevent further complications. Once such complication, which often occurs in light of a lack of fluids, is renal failure. Additionally, studies have suggested that a decrease in renal function is an early indicator to the failure of other vital organs, including the heart. Effective renal function depends upon glomerular filtration of serum into the renal tubules and selective tubular re-absorption and some renal tubular excretion. The glomerular filtration rate (GFR) is closely regulated by the constriction and dilation of afferent and efferent arterioles. Renal tubular function depends upon transmembrane pump mechanisms that affect the selective molecular passage against concentration gradients at a metabolic energy cost usually in the form of high energy phosphate compounds (e.g., adenosine triphosphate, or ATP). With physiologic stresses such as circulatory shock and overwhelming sepsis, energy supply and/or utilization is impaired, and consequently tubular function deteriorates. Without compensatory mechanisms, massive polyuria and uncontrollable hypovolemia may ensue, leading to further complications. However, intrinsic tubuloglomerular feedback mechanisms exist to limit volume losses in such states severely, primarily through the action of the macula densa at the juxtaglomerular apparatus. This appears to produce primarily an intrarenal release of renin producing afferent arteriolar constriction and probably some efferent arteriolar dilation, leading to a reduction in the filtration fraction, and a reduced GFR. This response occurs at the stage of tubular dysfunction and precedes the onset of acute tubular necrosis (ATN). Thus, close monitoring of the GFR provides a mechanism to detect renal compensation in a timely manner, allowing the potential for clinical interventions to reverse the physiologic stress and prevent decompensation in the form of ATN. It also appears that renal dysfunction and failure often precedes the onset of other or multiple organ failures. Thus, if renal function could be observed in near-real time, effective prevention of renal failure could potentially prevent the syndrome of multiple organ failure in many cases.

Other attempts to provide for monitoring a patient's GFR have focused upon the introduction of a marker substance, such as inulin, into the patient's bloodstream which is subsequently filtered out by the patient's kidneys at a measurable rate. The marker substance must be stable in the bloodstream and freely filtered by the kidneys without being reabsorbed nor secreted by the kidneys. However, these attempts have achieved limited success due to the requirement that a marker substance be administered into the bloodstream. Furthermore, the biosensors for detecting these substances with the required level of accuracy, both in the bloodstream and in the urine, are not cost effective. As such, a need exists for a system and method which will provide for a periodic measurement of the renal function of a patient in order to provide recommendations pertaining to the amount of fluid a patient should receive, and other potential interventions if necessary.

### SUMMARY

It is therefore an object of one embodiment of the present invention to provide a system for monitoring creatinine clearance rates in a patient, calculating proper intravenous fluid administration levels, and providing relevant feedback information and messages to the patient's physician, nurse, or other caregiver. A creatinine clearance rate below an established threshold results in the indication of a need for an intravenous fluid volume bolus, provided that the patient has not reach an established ceiling, while a creatinine clearance rate below a second threshold will generate a warning that requires additional measurement or caregiver intervention to insure the correct treatment is administered.

### BRIEF DESCRIPTION OF THE DRAWINGS

FIG. 1 is a diagrammatic view of a creatinine clearance monitoring and fluid level management system in accordance with one embodiment of the present invention.

FIG. 2 is a flowchart illustrating a process which controls the operation of a creatinine clearance monitoring and fluid level management system in accordance with one form of the present invention.

FIG. 3 is a flowchart illustrating a process which controls the operation of one step for determining a proper fluid infusion in the process of the creatinine clearance monitoring and fluid level management system of FIG. 2.

FIG. 4 is a flowchart illustrating a process which controls the operation of one step for determining in tubular function is present in the process of the creatinine clearance monitoring and fluid level management system of FIG. 2.

### DETAILED DESCRIPTION

For the purposes of promoting understanding of the principles of the invention, reference will now be made to the embodiments illustrated in the drawings and specific language will be used to describe the same. It will nevertheless be understood that no limitation of the scope of the invention is hereby intended and alterations and modifications in the devices, systems and representations illustrated in the drawings, and further applications of the principles of the present invention as illustrated herein being contemplated as would normally occur to one skilled in the art to which the invention relates.

While inulin clearance is a more commonly used method for measuring the glomerular filtration rate (GFR), a suitable stand-in is the determination of creatinine clearance. Creatinine is an endogenous molecule continuously released in the process of muscle metabolism, and therefore no infusion of a foreign substance is necessary for its determination. The creatinine clearance is determined from the following equation:

$$Ccr = \frac{Ucr \times V}{Pcr \times T}$$

Where C_{cr} is the creatinine clearance (usually in ml/min), U_{cr} and P_{cr} are the creatinine concentrations in urine and plasma respectively (usually in mg/dl), V is the volume of urine (usually in ml) collected over a period of time, T (in minutes). The normal C_{cr} is age dependent, averaging 125 ml/min in the prime of life and deteriorating to lower levels with aging.

There are two established uses of creatinine clearance determinations. The most common is its use as a monitor of renal function in patients with chronic renal insufficiency and failure. A 24-hour urine collection is obtained for this determination, providing a 24-hour average of the GFR estimate. Less commonly creatinine clearance is used as an assessment of renal function on an acute basis in critically ill individuals who are at risk of developing acute renal failure. Clinical studies have demonstrated a rapid renal response to physiologic stress with a sharp drop in C_{cr} (usually obtained over a 2-hour collection period). The drop in C_{cr}, if sustained, precedes the onset of renal insufficiency and subsequent acute renal failure. The drop in C_{cr} occurs in patients with normal systemic blood pressures and urinary flow rates, which are the indicators commonly monitored by current medical procedures to track fluid levels. The drop in C_{cr} usually precedes the oliguria that accompanies acute renal failure by some 16 to 18 hours. On the other hand, if the acute drop in C_{cr} reverses, no subsequent renal dysfunction is experienced.

Despite the clinical evidence of the value of C_{cr} monitoring in critically ill patients, clinicians rarely use it. In part, this is due to inadequate understanding of renal function and traditional medical practices. It has been standard practice for over four decades to monitor hourly urine output and serum creatinine. The concept that glomerular filtration can suffer acute changes that manifest themselves as oliguria and elevated creatinine several hours or sometimes days later is not a part of standard clinical knowledge. However, another major reason for the infrequent use of C_{cr} as a renal function monitor is that the timed collection of urine, transfer of the specimen to the laboratory for analysis, and calculation of the result is tedious and cumbersome. As such, it is the goal of the system described herein to provide an automated continuous read-out of the C_{cr} along with alarms/alerts and instructions presented as screen displays and/or electronically spoken phrases.

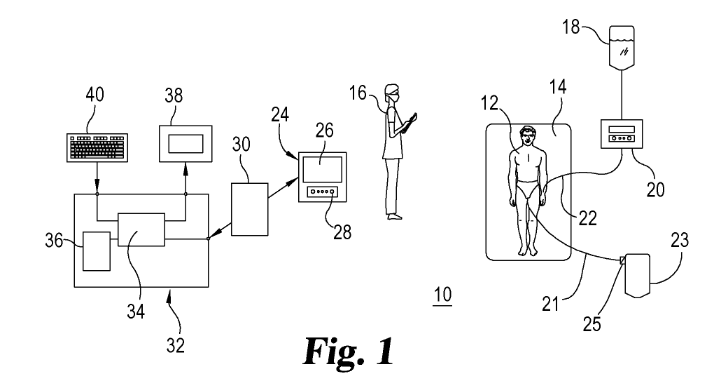

Referring to FIG. 1, there is shown an creatinine clearance monitoring and fluid level management system 10 for a patient 12 who is illustratively being cared for in a hospital critical care setting, e.g., within an intensive care unit following surgery, although other patient settings are of course possible. The condition and vital signs of patient 12 on bed 14 is shown as being illustratively monitored directly by a nurse or caregiver 16, but at least some functions that are performed by nurse 16 could be performed by automatic monitoring (pulse, blood pressure), data entry, and/or intravenous medication delivery equipment (not shown), to name only a few possible examples. For purposes of explaining an embodiment of the present invention, patient 12 is shown as receiving a continuous drip of fluid from reservoir 18 that is controlled by drip regulator 20 through an intravenous (IV) line 22. Additionally, patient 12 has a urinary catheter 21 in place which drains into a urine collection vessel 23, which includes a biosensor 25.

The system 10 includes various components for accurately determining and reporting the necessary elements to make an assessment of the patient's creatinine clearance. As discussed above, a proper C_{cr} calculation requires the input of a patient's creatinine concentrations in urine (U_{cr}) and creatinine concentrations in plasma respectively (P_{cr})(usually in mg/dl), in addition to the volume of urine (V)(usually in ml), and a time (T) over which that volume of urine was collected (in minutes).

The first element, time (T), is to be determined either statically by the user or dynamically by the system 10 based upon the user's criteria and is stored by data handling device 24. For example, the time element (T) may be defined within system 10 as any of a number of different intervals (e.g., 1 hour, 2 hours, 4 hours, 6 hours, 8 hours, 12 hours, 24 hours) and may be programmed so as to adapt automatically based upon the user's desires. This interval will set the cutoff for measurement of the volume (V) element to be used in the calculation of the C_{cr}. In addition, the T interval will be used to average all the U_{cr} values obtained during the prescribed interval, as described below.

In order to determine the patient's urine volume (V), the system utilizes catheter 21 and urine collection vessel 23. In operation, urine from patient 12 is carried via catheter 21 into collection vessel 23. In one form, catheter 21 is a Foley catheter, but it shall be appreciated that alternate catheter types may be utilized. According to another form, collection vessel 23 is a urimeter which is capable of determining and reporting urine volumes automatically. A suitable urimeter for use in this form is the CritiCore® Monitor manufactured by C.R. Bard, located at 730 Central Avenue, Murray Hill, N.J. 07974. This device is capable of accurate electronic monitoring of urine output using an embedded ultrasonic sensor. Furthermore, the urine volume can be collected and reported programmatically to data handling device 24, and interpreted in accordance with the timing intervals desired. Alternate methods of calculating the volume of urine output may be utilized, whether or not they capture the urine for measurement or estimate its volume based upon flow in discrete time intervals or the like.

According to the form illustrated, the system 10 utilizes a biosensor 25 in order to automatically collect the urinary concentration of creatinine (U_{cr}) of patient 12. The biosensor 25 may be one or more chemical field-effect transistors (chemFET), which periodically/continuously monitors the urinary concentration of creatinine (U_{cr}) in the urine passing through catheter 21 and reports its results to data handling device 24. A suitable type of chemFET for use as biosensor 25 is that described in “Creatinine Biosensors: Principles and Designs” by Anthony J. Killard and Malcolm R. Smyth, published in the Trends in Biotechnology Journal, Volume 18, Issue 10 (October 2000). Alternatively, other suitable biosensors known to those of skill in the art could be utilized as biosensor 25.

Finally, in order to determine the creatinine concentrations in plasma (P_{cr}), the nurse 16 orders a lab test based upon a blood sample drawn from patient 12. This is a lab test which nearly all critically ill patients at risk for renal failure have as a daily routine under current medical procedures. As such, the system 10 will be able to utilize this information without any additional burden on the hospital staff. Upon receiving the results of the P_{cr} value, the nurse 16 enters the P_{cr} value determined from the blood drawn from patient 12 into data handling device 24. Device 24 is illustratively shown as having a display 26 and an input interface 28. Display 26 may be of any conventional or available display type, such as, for example, a CRT or LCD screen, while input interface 28 may be a computer keyboard or touchscreen, for example. When the patient's P_{cr} test results have been entered into device 24, the entered information, along with the other values received (including T, V, and U_{cr}) are sent via communications channel 30 to computer or data processor 32 which may be located locally or at a central location, such as a nurse's station or hospital-wide patient monitor center. Communications channel 30 may be of the form of a hardwired connection, a local area network, a wireless network, or an internet-based wide area network, to cite a few non-limiting examples. A similar connection may exist between device 24 and one or more of urimeter 23, biosensor 25, and regulator 20. Network access may advantageously provide access to patient data from other hospitals or in-patient facilities, and it can allow patent 12 to be moved within a networked facility or between network-linked facilities, while still maintaining active monitoring of the patient's condition and providing access to historical patient data.

Data processor 32 illustratively comprises a central processing unit (CPU) 34 and memory 36, which may be of any known or available form, such as, for example, ROM, PROM, RAM, EPROM or EEPROM. Also shown as being connected or associated with data processor 32 are display 38 (such as, for example, a CRT, plasma, LED or LCD screen) and input device 40, such as a keyboard, for example.

Data processor 32 utilizes the creatinine concentration in urine (U_{cr}), creatinine concentration in plasma P_{cr})(usually in mg/dl), volume of urine (V)(usually in ml), and time (T) associated with patient 12, which were received from the data handling device 24, to calculate a continuous creatinine clearance rate (CCC) for patient 12. In the described form, this calculation is performed according to the equation described above. Additionally, the calculated CCC and other values, such as urine flow rate, may be determined and displayed on display 26 or some other in-room display or the like so as to be easily referenced by the attending medical staff. Additionally, prior to or at the outset of the CCC being monitored, the user is prompted to provide an Empiric Fluid Limit (EFL), which is the maximum amount of fluid that may be administered before an elevated evaluation is warranted for that patient in order to provide decision support. Such input may be provided by input interface 28.

Based upon the determine creatinine clearance rate (C_{cr}), along with additional variables, the system 10 will determine if the fluid levels of patient 12 are sufficient. If the patient's fluid levels are determined to be too low, data processor 32 will issue an alert and instruct the nurse 16 to administer a predetermined fluid intravenously to the patient 12. The information is sent back to device 24 via communications channel 30 where it appears on display 38. Nurse or caregiver 16 then makes any necessary adjustments to or initially sets up drip regulator 20 so that the proper amount of the selected fluid is delivered to patient 12 from reservoir 18. The calculation used by CPU 34 of data processor 32 illustratively utilizes a process which will be described below.

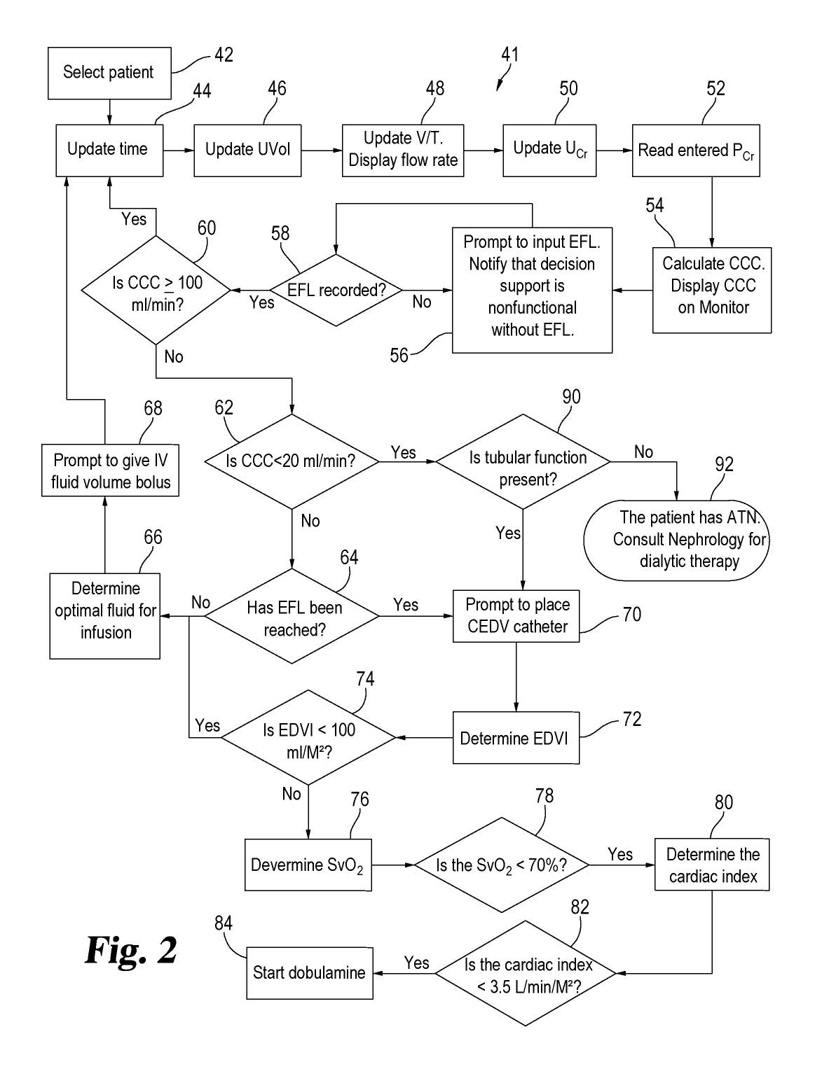

FIG. 2 illustrates a flowchart which, with continued reference to FIG. 1, shows a creatinine clearance monitoring and fluid level management process 41 in accordance with one form of the present invention, which will be used to illustrate the manner in which creatinine clearance monitoring and fluid level management system 10 of FIG. 1 may operate. Beginning at step 42 of process 41, a particular patient is selected for creatinine clearance monitoring and fluid level management by system 10. For illustrative purposes, we assume that the user or system 10 selects critical care patient 12 in step 42. Step 44 determines the current time and updates that information within the system 10. The system 10 then requests and receives the current urine volume (V) from urimeter 23 via data handling device 24 in step 46. Upon receiving this information, the system 10 updates the display of the current urine flow rate on display 38 in step 48. In continuing to collect the data necessary, the system 10 requests and receives the current creatinine concentrations in urine (U_{cr}) of patient 12 from biosensor 25 via data handling device 24 in step 50. In one form, this information is communicated to data collection device 24 electronically, such as via a wired or wireless connection. Additionally, in step 52 the system 10 receives the creatinine concentrations in plasma (P_{cr}) of patient 12. As discussed above, the P_{cr} value used by system 10 according to this form is a lab value determined from a sample of the blood of patient 12. As such, the P_{cr} value is entered into the system manually (such as via device 24) or through some other testing procedure sufficient to deliver an accurate P_{cr} value to system 10. It shall be appreciated by one of skill in the art that steps 46, 50, and 52 may occur simultaneously or in various order, with varying time intervals between steps also being possible.

Once system 10 has collected the necessary data, data processor 32 of system 10 calculates a continuous creatinine clearance rate (CCC) for patient 12 in step 54. This may occur once data handling device 24 has transmitted or made the received data accessibly to data processor 32. Once the CCC is calculated, it is preferably displayed on display 38 along with other information concerning patient 12. Moving on to step 56, the system 10 prompts the user to input the Empiric Fluid Limit (EFL) for patient 12. In this illustrated form, the EFL is input via device 24, such as by nurse 16 based upon the recommendation of the physician attending to patient 12. In alternate forms, the EFL for patient 12 may be provided earlier in process 41 or may be dynamically determined by system 10. Step 58 serves as a block which will prevent decision support of system 10 from being functional absent a specified EFL. As such, once an EFL is provided in step 56, the process 41 may advance to step 60. Alternatively, if the EFL has been entered previously, then step 58 is quickly satisfied and does not hold up process 41.

According to step 60, the data processor 32 of system 10 compares the CCC calculated in step 54 against a pre-determined threshold which represents the lower bound of a preferred range. This safe threshold may be determined based upon the sex, age, medical condition, and other characteristics of patient 12. For exemplary purposes, the threshold may be 100 mL/min and the system 10 may interpret a CCC greater than the 100 mL/min threshold to be satisfactory. As such, upon determining that the CCC of patient 12 determined in step 54 is above the first set threshold, process 41 iteratively returns to step 44 and begins a cyclic monitoring loop which repeatedly runs so long as the patient's CCC is above the safe threshold. However, if the system 10 determines that the CCC of patient 12 is below the desired threshold at any time then the process 41 proceeds to step 62. In step 62, system 10 determines whether the calculated CCC for patient 12 is below an emergency threshold, such as 20 mL/min.

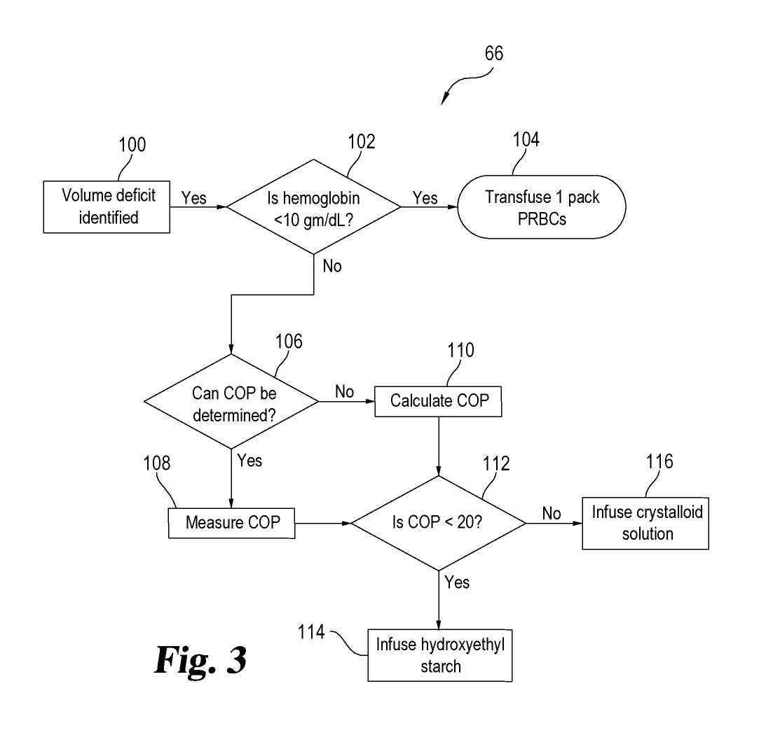

If the CCC of patient 12 is determined to be above the emergency threshold in step 62, then process 41 proceeds to step 64 in order to determine if additional fluids may be administered in order to attempt to raise the patient's fluid levels. As such, at step 64 a determination is made by system 10 as to whether or not the total volume of fluids given to patient 12 exceeds the EFL specified in step 56. In the event that it does not exceed the EFL, the process 41 proceeds to step 66 in which data processor 32 determines an optimal fluid order for infusion into patient 12. One exemplary process for use in making the determination in step 66 according to one form is described in detail below with reference to FIG. 3. Once the proper fluid infusion is determined, the system 10, such as through display 38, prompts the nurse 16 or other hospital staff to administer the determined fluid volume bolus to patient 12. Once the order is completed, the user indicates that the fluid order was filed and the process 41 returns to step 44 and begins anew. Additionally, the determined fluid volume provided to patient 12 is automatically added to the total volume of fluids administered to patient 12 for subsequent comparison against the EFL.

Returning to step 64, if the system 10 alternatively determines that the quantity of fluids administered to patient 12 exceeds the EFL, then the process advances to step 70 as the infusion of additional fluids has likely been previously attempted without success. At step 70, the system 10 prompts nurse 16 any other attending medical staff to effect the placement of a Continuous Right Ventricular End-Diagnostic (CEDV) catheter, or some other medically identified intervention. Additionally, in step 70, an alert may be issued to display 38 or otherwise, such as to the mobile device of nurse 16 or over the hospital's paging network. Once the CEDV catheter is in place, the system 10 receives a determination of the End Diastolic Volume Index (EDVI) in step 72. EDVI indicates the volume of blood in a ventricle at the end load or filling in diastole. An increase in EDVI increases the preload on the heart and, through the Frank-Starling mechanism of the heart, increases the amount of blood ejected from the ventricle during systole. Once the EDVI value is received, the system 10 compares the EDVI to an EDVI threshold, such as 100 ml/M². In the event the EDVI value of patient 12 is below the EDVI threshold, then process 41 returns to step 66 where a fluid infusion order is determined and the process continues from there. Alternatively, in the event the EDVI value of patient 12 is above the EVDI threshold, the process 41 proceeds to step 76 where the system 10 receives an SvO₂ value for patient 12. SvO₂ provides an assessment of total tissue oxygen balance (i.e., the relationship between oxygen delivery and oxygen consumption). SvO₂ varies directly with cardiac output, Hb, and SaO₂, and inversely with VO₂ (oxygen consumption). The normal SvO₂ is 75%, which indicates that under normal conditions, tissues extract 25% of the oxygen delivered. An increase in VO₂ or a decrease in arterial oxygen content (SaO₂×Hb) is compensated by increasing CO or tissue oxygen extraction. When the SvO₂ is less than 30%, tissue oxygen balance is compromised, and anaerobic metabolism ensues. As such, in the step 78 the system 10 determines if the SvO₂ of patient 12 is below a SvO₂ threshold, such as 70%. If the SvO₂ is greater than 70%, then the process 41 ends at this point and, in one form, would restart by returning to step 44 as it is intended to be a continuous monitoring process.

In the event that the SvO₂ value is below this threshold, the process proceeds to step 80 in which the system prompts for and receives a cardiac index associated with patient 12. Cardiac index (CI) is a vasodynamic parameter that relates the cardiac output (CO) to body surface area (BSA), thus relating heart performance to the size of the individual. The unit of measurement is liters per minute per square meter (l/min/m²). The process 41 proceeds to step 82 in which the cardiac index of patient 12 is compared to a cardiac index threshold, such as 3.5 l/min/m². In the event that the cardiac index of patient 12 is below this threshold, the system 10 provides an alert, such as via display 38, instructing that dobutamine be administered to patient 12. Dobutamine is a sympathomimetic drug used in the treatment of heart failure and cardiogenic shock. Its primary mechanism is direct stimulation of β₁ receptors of the sympathetic nervous system.

Returning to step 62, if the CCC of patient 12 is determined to be below the emergency threshold, then process 41 proceeds to step 90 which questions the medical staff as to whether tubular function is present. In the event that tubular function is prevent, the process 41 proceeds to step 70 and proceeds accordingly. However, in the event tubular function is not determined to be present, upon being notified, the system 10 instructs the attending medical staff to consult with the nephrology department as the patient has acute tubular necrosis (ATN) which requires dialytic therapy. In such an event, the process 41 ends at this point.

Turning to FIG. 3, with continued references to FIGS. 1 and 2, a flowchart is illustrated showing the detail of step 66 of FIG. 2 in which a fluid determination is made according to one embodiment of system 10. The process of step 66 begins at start point 100 with a fluid deficiency being identified. The process proceeds to step 102 in which a determination is made as to whether the hemoglobin level of the patient 12 is less than a hemoglobin threshold, such as 10 gm/dL. If the hemoglobin level is less that the hemoglobin threshold, then the process proceeds to step 104 in which determination is made that 1 pack of Packed Red Blood Cells (PRBCs) be transfused into patient 12. Upon reaching step 102, the process of step 66 returns to step 68 where an alert is issued according to the interventions determined to be needed by system 10. Alternatively, if the hemoglobin level is greater than the hemoglobin threshold, then the process proceeds to step 106 in which system 10 seeks the patient's colloid osmotic pressure (COP). The COP is a pressure normally exerted by proteins in blood plasma that usually tends to pull water into the circulatory system. In the event the COP can be readily determined, the process moves to step 108. Alternatively, in the event the COP is not readily determinable, the process moves to step 110 to calculate a COP for patient 12. No matter the path, the process of step 66 arrives at step 112 where the patient 12's COP is compared to a COP threshold. For exemplary purposes, this COP threshold is set at 12. In the event the patient's COP is below 12, the process proceeds to step 114 in which the system 10 recommend the infusion of hydroxyethyl starch for patient 12. Alternatively, when the patient's COP is above 12, the process proceeds to step 116 in which the system 10 recommend the infusion of a crystalloid solution for patient 12. Upon reaching either step 114 or 116, the process of step 66 returns to step 68 where an alert is issued according to the interventions determined to be needed by system 10.

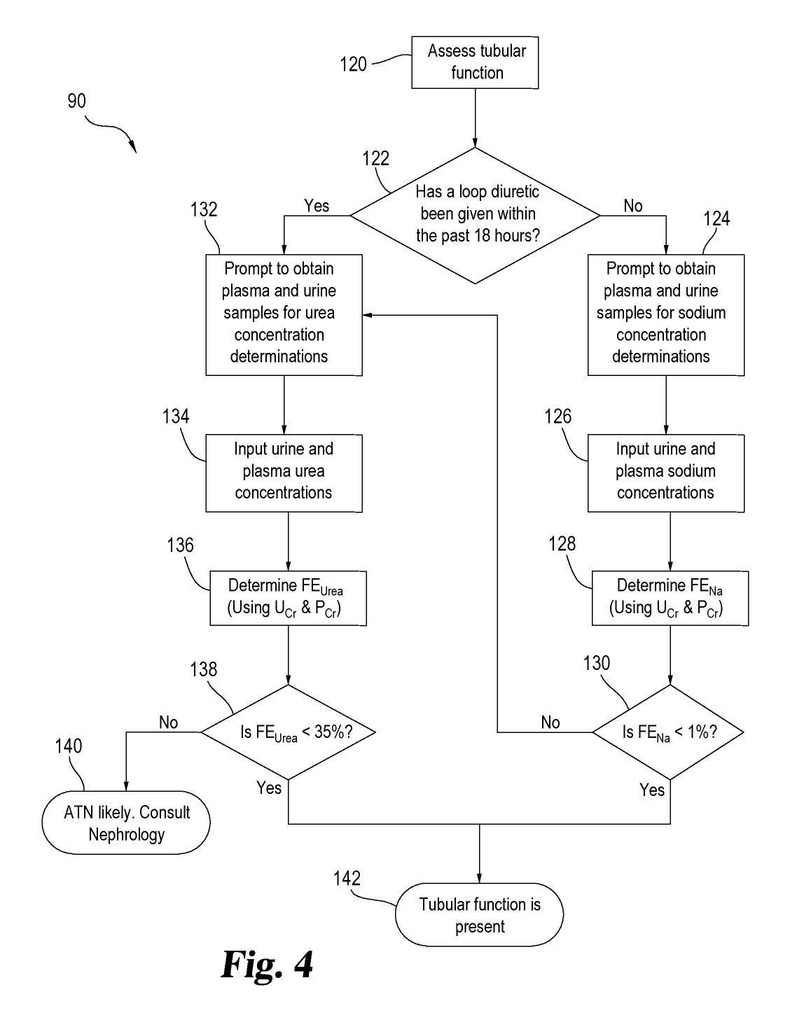

Turning to FIG. 4, with continued references to FIGS. 1 and 2, a flowchart is illustrated showing the detail of step 90 of FIG. 2 in which a determination is made as to whether or not tubular function is present within patient 12 according to one embodiment of system 10. The process of step 90 begins at start point 120 with the need to assess the tubular function of patient 12 being identified. The process proceeds to step 122 in which it is determined whether the patient has received a diuretic in the past 12 hours. If the patient 12 has not been given a diuretic, then the process proceeds to step 124 in which the system 10 prompts the nurse 16 to obtain plasma and urine samples from patient 12 for sodium concentration testing. Subsequently, the process proceeds to step 126 where the nurse 16 is requested to input plasma and urine sodium concentrations based on the assay results from the samples taken in step 124. The process then proceeds to step 128 where a fractional excretion of sodium (FE_{Na}) for patient 12 is determined using the known U_{cr} and P_{cr} for patient 12. FE_{Na} is the percentage of the sodium filtered by the kidney which is excreted in the urine. If the determined FE_{Na} is less that a FE_{Na} threshold, such as 1%, the process proceeds to step 142 where the presence of tubular function for patient 12 is indicated. Alternatively, if the determined FE_{Na} is greater than the threshold, the process proceeds to step 132 where the process rejoins the outcome where patient 12 has been given a diuretic in the past 12 hours from step 122. From here, in step 132, the system 10 prompts the nurse 16 to obtain plasma and urine samples from patient 12 for urea concentration testing. Subsequently, the process proceeds to step 134 where the nurse 16 is requested to input plasma and urine urea concentrations based on the assay results from the samples taken in step 132. The process then proceeds to step 136 where a fractional excretion of urea (FE_{Urea}) for patient 12 is determined using the known U_{cr} and P_{cr} for patient 12. FE_{Urea} is the percentage of the urea filtered by the kidney which is excreted in the urine. If the determined FE_{Urea} is less that a FE_{Urea} threshold, such as 35%, the process proceeds to step 142 where the presence of tubular function for patient 12 is indicated. Alternatively, if the determined FE_{Na} is greater than the threshold, the process proceeds to step 140 where the system 10 indicates the presence of acute tubular necrosis (ATN). Upon reaching step 140 the process of step 90 returns to the process 41 of FIG. 2 at step 92. Alternatively, upon reaching step 142 of the process of step 90 returns to the process 41 of FIG. 2 at step 70 for subsequent handling by stem 10.

It shall be appreciated that many of the functions of system 10 that have been described with reference to FIGS. 2-4 may be performed by electronic circuitry and/or with computer software, including but not limited to the steps of retrieving values, calculating creatinine clearance rates, determining whether calculated amounts are above specified thresholds, issuing alert messages, and determining fluid volume bolus orders.

The previous description has been made based on treatment of patients in an in-patient medical/surgical setting, such as a hospital or nursing home, as the novel features of the invention lend themselves particularly well to a critical or intensive care setting. The scope of the invention, however, is not limited to an in-patient environment. Significant advantages can also be realized by ambulatory or otherwise medically attendant individuals with a need for fluid level monitoring through the use of, for example, periodic infusions. The manner in which such as system, incorporating one or more embodiments of the present invention, could provide automatic tests and administration of proper infusion amounts while still maintaining sufficient safeguards to protect against an inadvertent application of an incorrect dose due to an equipment malfunction or some incident of human error.

While the invention has been illustrated and described in detail in the drawing and foregoing description, the same is to be considered as illustrative and not restrictive in character, it being understood that only the preferred embodiment has been shown and described and that all changes, modifications and equivalents that come within the spirit of the inventions disclosed are desired to be protected. The articles “a”, “an”, “said” and “the” are not limited to a singular element, and include one or more such elements.

What is claimed is:

1. A system for monitoring fluid levels in an individual with respect to a desired threshold and prompting when intervention is required comprising:
an electronic network;
an electronic database connected to said network, said database being suitable for storing a user input range of desired results for creatinine clearance rate of the individual;
a urimeter connected to the patient via a catheter capable of calculating a volume of urine contained therein and reporting the same electronically to said database over said network;
a biosensor positioned between said urimeter and the patient which is capable of determining a creatinine concentration in urine result for the individual; and
a user interface suitable for receiving a creatinine concentration in plasma assay result obtained based on the blood of the individual;
one or more computer processors connected to said network,
wherein said computer processors are responsive to said creatinine concentration in urine, said creatinine concentration in plasma, and said volume of urine to determine a creatinine clearance rate, and
wherein said computer processors are responsive to said creatinine clearance rate to determine a fluid bolus infusion for the individual when said creatinine clearance rate is below said desired threshold.

2. The system of claim 1, wherein said urimeter and said biosensor operate on a periodic basis.

3. The system of claim 1, wherein said urimeter and said biosensor operate on a continuous basis.

4. The system of claim 1, wherein said computer processors are further responsive to at least one criteria associated with the individual.

5. The system of claim 1, wherein said criteria comprises previous creatinine clearance rates associated with the individual.

6. The system of claim 1, wherein said criteria comprises the total volume of fluids administered to said individual in a defined period and a user specified fluid limit volume.

7. The system of claim 1, wherein said criteria comprises information regarding a previous fluid volume bolus infusion.

8. The system of claim 1, wherein said fluid bolus infusion is a crystalloid solution.

9. The system of claim 1, wherein said fluid bolus infusion is hydroxyethyl starch.

10. The system of claim 1, wherein said individual is a patient in a hospital.

11. The system of claim 1, further comprising notification means for generating an alert signal when said creatinine clearance rate is below said threshold.

12. The system of claim 11, wherein said alert signal is auditory.

13. The system of claim 11, wherein said alert signal is visual.

14. A method for monitoring fluid levels in an individual with respect to a desired threshold and prompting when intervention is required comprising the steps of:
establishing a desired lower threshold of a creatinine clearance rate for the individual;
receiving a creatinine concentration in plasma assay result for the individual;
storing said creatinine concentration in plasma assay result in an electronic database;
automatically sensing a creatinine concentration in urine result for the individual using a biosensor in fluid communication with a catheter connected to the patient;
storing said creatinine concentration in urine result in said database;
determining a urine volume for a specified time internal using a urimeter;
storing said urine volume in said database;
calculating a creatinine clearance rate for the individual using one or more computer processors and aid creatinine concentration in urine result, said creatinine concentration in plasma result, and said urine volume;
comparing said creatinine clearance rate to said desired threshold in response to said calculating, using said one or more computer processors; and
determine a fluid bolus infusion for the individual using said one or more computer processors when said creatinine clearance rate is below said desired threshold.
